(19) United States
(12) Patent Application Publication
(10) Pub. No.: US 2026/0226385 A1
(43) Pub. Date: Aug. 6, 2026

(54) CELL-CULTURE INSERT FOR CULTIVATING CELLS

(71) Applicant: BRAND GMBH + CO KG, Wertheim (DE)

(72) Inventors: Stefan MATREUX, Triefenstein (DE); Philipp NICOLA, Volkach (DE); Thorsten RAWINSKI, Marktheidenfeld (DE); Rhea SPAIIR, Mosbach (DE); Michael GEISLER, Offenbach am Main (DE); Peter KRANK, Wertheim (DE); Nicolas FRANK, Würzburg (DE)

(21) Appl. No.: 19/151,404

(22) PCT Filed: Jan. 18, 2024
(86) PCT No.: PCT/EP2024/051155

§ 371 Date: Jul. 28, 2025

(30) Foreign Application Priority Data
Jan. 26, 2023 (DE) 20 2023 100 368.0

(51) Int. Cl. C12M 1/12; C12M 1/32; C12M 3/00

(52) U.S. Cl. CPC C12M 25/04; C12M 23/12; C12M 23/44; C12M 23/48

(57) ABSTRACT

A cell culture insert for cultivating cells, having a side wall (7) which encloses a cavity (11) and has an inner face (8) delimiting the cavity (11) and an outer face (9), preferably wherein the side wall (7) forms a hollow cylinder and/or surrounds the cavity (11) substantially in the shape of a hollow cylinder, a bottom designed as a membrane (14) and a fastening element (18), wherein the membrane (14) is fastened to the inner face (8) of the side wall (7) by the fastening element (18) in a force-fit and/or form-fit manner.

### BACKGROUND OF THE INVENTION

### Field of the Invention

The invention relates to a cell culture insert for cultivating cells having a side wall which encloses a cavity and comprises an inner face, which delimits the cavity, an outer face, a bottom which is a membrane, and a fastening element.

### Description of Related Art

Cell culture inserts of the type in question are regularly used in so-called well plates. The well plate contains several wells for holding liquid. The wells are usually molded into the well plate. Each well has a bottom, a surrounding wall that rises up from it and a rim that ends the surrounding wall at the top. The rim defines the front access opening into the well.

Liquid is poured into the wells of the well plate. The liquid serves as a nutrient fluid for the cells, which are cultivated on the bottom of the well or on the bottom of a cell culture insert inserted into the well. The nutrient liquid is added to the well and changed using a pipette.

Cell culture inserts of the type in question are therefore used to cultivate cells on the bottom of the cell culture inserts, which is designed as a membrane. At least one support arm projecting laterally from the upper opening rim of the respective cell culture insert is regularly used to suspend the cell culture inserts in the wells. Cultivating cells in the hanging insert at a relatively large distance from the bottom of the well allows sufficient nutrient fluid to be added to the well and around the cell culture insert.

However, there are also cultivation methods in which the cell culture inserts should be placed on a support. For this purpose, it is known to provide support feet projecting downwards from the rim of the side wall in addition to or as an alternative to a support arm. These can be used to set up the cell culture insert on a support with a small, even distance between a bottom and the support so that it cannot tip over.

The bottom of the cell culture insert is regularly designed as a microporous membrane. The membrane enables an exchange with the nutrient liquid under the bottom of the insert if the cell culture insert is arranged in a well of a well plate filled with nutrient liquid. Membranes for cell culture inserts can be made of plastic, such as PTFE, PE, PET, MCE and/or PC. Collagen membranes are also increasingly being used. Collagen is a structural protein of the extracellular matrix and is found, for example, in human and animal skin, bones, muscles, tendons, cartilage or connective tissue. Structural proteins are proteins that primarily serve as scaffolding materials in the tissues or cells of living organisms. Collagen is the most abundant protein in human and animal bodies and accounts for 25% to 30% of human and animal protein.

One possible application of collagen membranes is the cultivation of adherent and non-adherent cells, primary cells, stem cells and cell lines and the construction of tissue structures. A cell-colonized collagen membrane can be embedded directly to create a histological section, whereby the collagen membrane can also be stained. The collagen membrane is permeable to nutrients, which enables further applications such as the cultivation of cells for the construction of skin models or the performance of transmembrane transport studies.

In the prior art on which the invention is based (DE 20 2017 003 978 U1), the cell culture insert has a side wall that encloses a cylindrical cavity. The membrane is disk-shaped and has a diameter essentially corresponding to the outer diameter of the side wall. To create a connection between the side wall and the membrane, the side wall is glued to the membrane at the end face.

When using membranes made of a biological material, such as collagen, gaps can occur between the membrane and the side wall if the membrane is damaged by the adhesive and/or the bonding process, as a result of which the membrane can partially detach from the side wall. The mechanical properties and permeability of the membrane can also be negatively affected in places by the adhesive. It must also be ensured that the adhesive and the bonding process used are not toxic to the cells, which can have a negative impact on cell growth.

Alternatively, it is also known from the state of the art to connect the membrane to the side wall with a material bond. For this purpose, the side wall can at least in sections have a material, such as plastic, which has a lower softening temperature than the membrane. Heating the material can cause it to liquefy and penetrate into the pore spaces of the membrane, creating undercuts. After the material has cooled and hardened, there is a positive connection between the side wall and the membrane.

High temperatures are required to create a positive connection between the side wall and the membrane by softening the material of the side wall. It has been shown that a membrane consisting of collagen or containing collagen can be damaged by the high temperatures required, which can negatively affect cell growth.

### SUMMARY OF THE INVENTION

The present invention is directed to the problem of creating a cell culture insert that enables an easy-to-establish connection between the side wall and the membrane, while ensuring a reliable connection between the membrane and the side wall. The task of the invention is also to ensure that the membrane remains flat in the cell culture insert at all times to prevent bulging or sagging of the membrane.

The above problem is solved in a cell culture insert with the features of claim 1. Preferred embodiments and further developments are the subject of the subclaims directed to the cell culture insert.

According to the invention, the cell culture insert has a side wall that encloses a cavity, with an inner face delimiting the cavity and an outer face. It is preferably provided that the side wall forms a hollow cylinder and/or surrounds the cavity in a substantially hollow cylindrical shape. The cell culture insert also has a bottom designed as a membrane and a fastening element, whereby the membrane is attached to the inner face of the side wall by means of the fastening element in a force-fit and/or form-fit manner. The non-positive and/or positive attachment of the membrane to the inner face of the side wall by means of the fastening element enables particularly simple handling and easy assembly of the cell culture insert. The cell culture insert can therefore be manufactured particularly cost-effectively. Due to the force-fit and/or form-fit fastening of the membrane to the inner face of the side wall, it is not necessary for the fastening element to protrude beyond the side wall in the radial direction, which means that the installation space required for the cell culture insert can be kept to a minimum.

The membrane can be attached to the side wall via the fastening element in various ways, as explained below. For example, it can be particularly useful if the membrane is arranged in the cavity of the side wall. The membrane is then shielded in the radial direction by the side wall when the cell culture insert is mounted, which can prevent unintentional damage to the membrane.

It is particularly advantageous if the membrane has an external shape that complements the design of the inner face of the side wall. The membrane can then be positioned relative to the side wall in a particularly simple manner.

The simple manufacture of the membrane is of great importance for cost reasons. For this purpose, the membrane can be designed as a cylinder or cylindrical or disk-shaped. The separation of a correspondingly shaped membrane from a rectangular template, for example, is then possible in a particularly simple manner, in particular by machine, for example by means of a punching process.

In particular, the membrane can have a radially outer rim portion. In the assembled state of the cell culture insert, the rim portion is preferably arranged between the inner face of the side wall and an outer face of the fastening element.

Alternatively or additionally, the membrane can be arranged in the assembled state at least essentially at the level of a bottom rim delimiting the front of the cavity.

A wide range of application of the membrane can also be achieved if the membrane is formed from at least one organic material. It is particularly preferable for the membrane to be made of collagen. The membrane then has biocompatible properties and therefore enables a large number of possible applications, which are mentioned by way of example in the introduction to the description.

In an alternative embodiment, the membrane can be made of a material that cannot be bonded to the side wall. This can be the case, for example, with materials that are particularly temperature-sensitive and whose mechanical properties and permeability can be negatively affected by correspondingly high temperatures. This also applies to materials whose mechanical properties and permeability can be negatively affected by the use of suitable adhesives.

The arrangement and design of the fastening element is of great importance for the simple formation of a connection between the membrane and the side wall. For example, it is possible for the fastening element to be arranged at least partially in the cavity, thereby achieving a compact structure of the cell culture insert. The fastening element can alternatively or additionally project outwards beyond the side wall and in this way provide a minimum distance between the side wall of the cell culture insert and a circumferential wall of a well of the well plate.

Reliable fastening of the membrane to the side wall can be achieved by the fastening element having a pressing force acting radially outwards on the side wall. Alternatively or additionally, the fastening element can form an interference fit with the side wall. The term “interference fit” is generally understood to mean that the inner maximum dimension of the receiving component is always smaller than the outer minimum dimension of the received component. Press fits can be produced cost-effectively and at the same time represent a secure and defined type of connection.

The costs for the cell culture insert can be further reduced if the fastening element is designed as a hollow cylinder, preferably with a rectangular or conical longitudinal section. Compared to a disk-shaped or cylindrical fastening element, material savings can be achieved in this way. In order to keep the complexity of the cell culture insert low, it is alternatively or additionally provided that the fastening element is designed as a single piece.

A particularly strong connection between the fastening element and the side wall can be achieved if the fastening element has a roughened surface on one outer face.

It is particularly useful for securely fastening the membrane to the side wall if the fastening element has an outer diameter that is larger than the diameter of the membrane. It is also useful for secure and defined support of the membrane against the fastening element if the fastening element alternatively or additionally has an inner diameter that is smaller than the diameter of the membrane.

The mechanical stability of the side wall and the fastening element is of crucial importance for securely fastening the membrane to the side wall. A particularly stable fastening element that is also inexpensive to manufacture can be obtained if the fastening element has polypropylene, polyethylene, polystyrene and/or polylactide acid or consists of polypropylene, polyethylene, polystyrene and/or polylactide acid. The fastening element can then also be disposed of cost-effectively.

To reduce the variety of materials, it is particularly advantageous if the fastening element is made of the same, in particular biocompatible, material as the side wall of the cell culture insert.

A particularly reliable connection between the fastening element and the side wall can be achieved if the side wall forms a hollow truncated cone. Alternatively or additionally, the side wall can surround the cavity essentially in the shape of a hollow truncated cone. The cavity is then preferably designed as a truncated cone and/or truncated cone-shaped.

The fastening element is preferably formed on an outer face complementary to a section of the side wall delimiting the cavity, in particular the inner face of the side wall. In particular, the fastening element is pressed into the side wall. Preferably, a press fit is provided between the side wall and the fastening element.

The inner face of the side wall preferably has a top face against which the membrane rests in the fastened state.

In order to fasten the membrane to the side wall in a particularly reliable manner, according to a preferred teaching of the invention, it is provided that a preferably circumferential stop for fastening the membrane is formed on the inner face of the side wall, the fastening element is pressed into the stop and the fastening element holds the membrane or presses it against the stop.

The membrane preferably has a top face and a bottom face, whereby the membrane preferably rests against the stop only with a section of its top face. A section of the bottom face of the membrane can rest against the fastening element. The membrane is then held between the fastening element and the stop, in particular in the axial direction, and is thus positively and/or non-positively fastened to the inner face of the side wall.

Alternatively, it is also possible for the top face of the membrane to rest against the stop and the bottom face to rest against the fastening element, whereby the membrane is held in a force-fit and/or form-fit manner between the fastening element and the side wall. The outer face of the side wall can have an external thread for connection to the fastening element, which engages with an internal thread on an inner face of the fastening element when the cell culture insert is mounted. To attach the membrane to the side wall, it is then possible to simply place the fastening element on the side wall and screw it directly to it. Alternatively, it is also possible for the inner face of the side wall to have an internal thread and the fastening element to have an external thread on an external side, which is engaged or can be engaged with the internal thread of the side wall.

A particularly practical design of the side wall is one in which the stop is formed by a recess on the inner face of the side wall and has two preferably circumferential stop surfaces arranged at an angle, in particular at right angles, to each other. It is particularly advantageous if one stop surface runs at least substantially in the radial direction and in the circumferential direction and the other stop surface runs at least substantially in the axial direction and in the circumferential direction. In this way, a simple arrangement of the membrane within the cavity relative to the side wall and a simple arrangement of the membrane between the fastening element and the side wall can be realized.

A permanently flat arrangement of the membrane in the fixed state of the cell culture insert without curvature of the membrane can be ensured if the membrane rests against one stop surface and is spaced from the other stop surface. It is particularly advantageous if the membrane rests against the stop surface running essentially in the radial direction and in the circumferential direction and is spaced apart from the stop surface running essentially in the axial direction and in the circumferential direction. The membrane, in particular the outer rim of the membrane, is then at least partially spaced from the inner face of the side wall. If, for example, contact of the membrane with nutrient fluid causes the membrane to expand, in particular in the radial direction, the membrane can expand in the radial direction until the membrane is in full contact with the inner face of the outer wall without causing the membrane to bulge. Accordingly, the distance between the inner face of the side wall and the membrane can be specifically adjusted to ensure that the membrane is kept free of curvature in the radial direction at all times. The membrane then rests with its bottom face on the fastening element and with its top face on the stop and is fixed in a first axial direction and in the radial direction by the stop and in the second axial direction by the fastening element.

A design of the cell culture insert according to the invention is particularly useful, in which the stop has at least one recess on the stop surface extending essentially in the axial direction and in the circumferential direction and the fastening element has at least one complementary protrusion on an outer face and in which the recess and the protrusion are in engagement or form a positive fit in the fastened state. In this way, a particularly secure connection can be obtained between the side wall and the fastening element. By engaging the protrusion in the recess, the correct positioning of the fastening element relative to the side wall can be easily checked, for example visually or by a sudden drop in the force required to create the connection. In this way, the operational safety of the cell culture insert can be increased and unintentional detachment of the fastening element from the side wall can be reliably prevented.

Alternatively, or additionally, it is also possible for the stop to have at least one recess on the stop surface running essentially in the radial direction and in the circumferential direction and for the fastening element to have at least one protrusion on one end face, with the protrusion engaging at least in sections in the recess and deforming the membrane in the axial direction, thereby creating a radial tension on the membrane in order to further promote a wrinkle-free and smooth plane.

It is also possible for the stop to be formed by a clamping protrusion on the inner face of the side wall. The clamping protrusion can be conical in longitudinal section and/or form a circumferential stop surface against which the membrane, in particular the bottom face, rests. The stop surface runs in particular transversely to the radial and/or axial direction. In this way, a wrinkle-free and curvature-free arrangement of the membrane can be realized. At the same time, a reliable force-fit connection can be formed between the fastening element and the stop on the clamping protrusion or additionally in the radial direction on the inner face of the side wall.

It is also possible that the circumferential stop surface, which is formed transversely to the radial direction, has at least one recess and the fastening element has at least one complementary protrusion on an outer face. In the fastened state, the protrusion can engage in the recess and in this way form a positive fit. In this way, a particularly secure connection can be obtained between the side wall and the fastening element. By engaging the protrusion in the recess, the correct positioning of the fastening element relative to the side wall can be easily checked, for example visually or by a sudden drop in the force required to create the connection.

In order to ensure that the nutrient fluid can only penetrate through the membrane into the cavity of the cell culture insert, in an appropriate embodiment of the cell culture device it is provided that the fastening element and/or the stop of the side wall have grooves or a sealing lip, whereby a particularly reliable seal is formed between the fastening element and the side wall.

The reusability of individual components of the cell culture insert can be achieved by detaching the fastening element and the membrane from the side wall. It is then also possible to replace the membrane and in this way adapt the cell culture insert specifically to the respective application. Due to the force-fit and/or form-fit attachment of the membrane to the inner face of the side wall, it is not necessary to remove adhesive residue from the side wall and/or the membrane, which makes it easier to detach the membrane from the side wall and to replace the membrane.

For easy detachment of the membrane from the side wall, it may be provided that the fastening element has at least one externally accessible indentation for removal by means of a tool. In this way, an optimized flow of force can be generated on the fastening element or a lever arm can be created, making it particularly easy to detach the fastening element from the side wall.

In an alternative embodiment, the fastening element is ferromagnetic for removal by means of a magnetic device. With the aid of a magnet, for example a permanent magnet or an electromagnet, a force can be exerted on the fastening element by which the fastening element can be released from the side wall. Alternatively, it is also possible for the fastening element to have at least one protrusion projecting radially inwards from an inner face of the fastening element with an externally accessible indentation for removal by means of a tool. The protrusion can be spaced from a rim of the inner face of the fastening element.

The assembly of the cell culture insert can be further simplified if the fastening element is made in several parts, preferably in two parts, and the membrane is clamped between parts of the fastening element. For example, the fastening element can consist of two hollow cylindrical elements between which the membrane is clamped. It is possible, for example, that the two parts of the fastening element can be connected to each other in such a way that the membrane is clamped between the two parts and the two fastening elements and the membrane are fixed relative to each other. In this way, a pre-assembled functional unit consisting of the fastening element and the membrane is created. The functional unit consisting of the membrane and the fastening element can then be connected to the side wall in a single assembly movement.

Alternatively, it is also possible to design the fastening element in several parts, preferably in two parts, whereby the membrane is clamped between at least one part of the fastening element and the side wall. For example, the fastening element can consist of two identically designed elements which, when placed together in the circumferential direction, essentially form a hollow cylinder. It is also possible for the individual parts of the fastening element to be connected to each other, for example, to be latched together and/or positively connected. By using two fastening elements, it is possible to divide the membrane into an area accessible to cells and an area not accessible to cells or to treat areas of the membrane differently and use them for subsequent comparative tests.

A design in which the fastening element has a support device arranged below the membrane is particularly preferred. The support device ensures that the fastening element is stiffened, particularly in the radial direction. The support device can rest against the bottom face of the membrane, which can prevent the membrane from bulging or sagging in the axial direction.

In order to position the cell culture insert on a bottom, in particular a well plate, so that it cannot tip over, at least three support elements can be attached to a bottom face of the fastening element for positioning the cell culture insert so that it cannot tip over and for removing the fastening element. It is then possible, for example, to provide different fastening elements with differently shaped support elements for cell culture inserts, so that a fastening element with corresponding support elements can be selected depending on the application.

The cell culture insert according to the invention enables a modular structure in which a side wall can be combined with different membranes and/or different fastening elements in a particularly simple manner. In this way, the cell culture insert can be used in a variety of ways and can be adapted to the respective application in a simple and cost-effective manner by selecting a suitable membrane and a suitable fastening element.

Assembly of the cell culture insert is particularly simple. The membrane is first connected to the fastening element or placed against the stop of the side wall. The fastening element can then be inserted into the receiving space defined by the stop and pressed against the stop, whereby the membrane is fastened, in particular pressed, to the inner face of the outer wall in a force-fit and/or form-fit manner.

The invention also relates to a device for cultivating cells with a well plate and a cell culture insert according to one of claims 1 to 16.

In the following, the invention will now be explained in more detail with reference to the drawings showing preferred embodiments of the invention.

### BRIEF DESCRIPTION OF THE DRAWINGS

FIG. 1 shows a perspective view of a device according to the invention for cultivating cells with a well plate with several, here a total of twenty-four, wells for holding liquid, and with several, here a total of four, cell culture inserts according to the invention,

FIG. 2 shows a perspective view from above of a cell culture insert according to the invention from FIG. 1,

FIG. 3 shows a perspective view from below of the cell culture insert according to the invention shown in FIG. 2,

FIG. 4 shows the cell culture insert from FIG. 2 in a longitudinal section,

FIG. 5 shows a perspective view of a longitudinal section of the side wall and the fastening element of FIG. 4 in a first embodiment,

FIG. 6 shows a perspective view of a longitudinal section of the side wall and the fastening element from FIG. 4 in a further embodiment,

FIG. 7 shows a perspective view of a longitudinal section of the side wall and the fastening element from FIG. 4 in a further embodiment,

FIG. 8 shows a longitudinal section of the side wall and the fastening element from FIG. 4 in a further embodiment,

FIG. 9 shows a longitudinal section of a cell culture insert according to the invention from FIG. 1 according to a further embodiment,

FIG. 10 shows a longitudinal section of a cell culture insert according to the invention from FIG. 1 according to a further embodiment,

FIG. 11 shows a perspective view obliquely from below of the cell culture insert of FIG. 2 in a further embodiment with a fastening element which has two removal elements,

FIG. 12 shows in a perspective view oblique from below, the cell culture insert of FIG. 2 in a further embodiment with a fastening element comprising a support device,

FIG. 13 shows in a perspective view oblique from above, the fastening element of FIG. 12 and

FIG. 14 shows a perspective view of the cell culture insert from FIG. 2 at an angle from below in a further embodiment with a fastening element that has support elements.

### DETAILED DESCRIPTION OF THE INVENTION

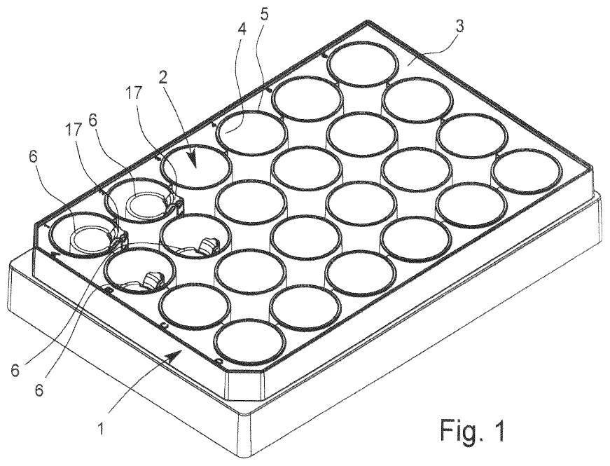

FIG. 1 shows a particularly preferred example of a device for cultivating cells. This has a well plate 1, which is made of thermoplastic material. In the well plate 1 there are several wells 2, in this case a total of twenty-four, for holding liquid. The well plate 1 has an upper face 3 extending over part of the surface, on which, as shown in FIG. 1, the identification numbers are located. The wells 2 are integrally molded into the well plate 1. In the spaces between wells 2, the upper face 3 is recessed so that lateral walls 4 of the wells 2 can be seen because they are freely accessible.

Each well 2 has a bottom portion, not shown in detail in FIG. 1, from which the upstanding, circumferential side wall 4 extends to a rim 5 terminating at the top. In FIG. 1, it is easy to see from the empty wells 2 on the right that each well 2 has a specific lateral internal dimension in a plane parallel to the bottom portion and a specific internal height in the direction perpendicular to the bottom portion. In the example shown, the wells 2 are cylindrical so that the lateral internal dimensions are defined by a specific internal diameter. Non-circular wells 2 have more complex lateral internal dimensions. FIG. 1 shows a cell culture insert 6 in each of the wells 2 with the alphanumeric numbering A1, A2, B1 and B2.

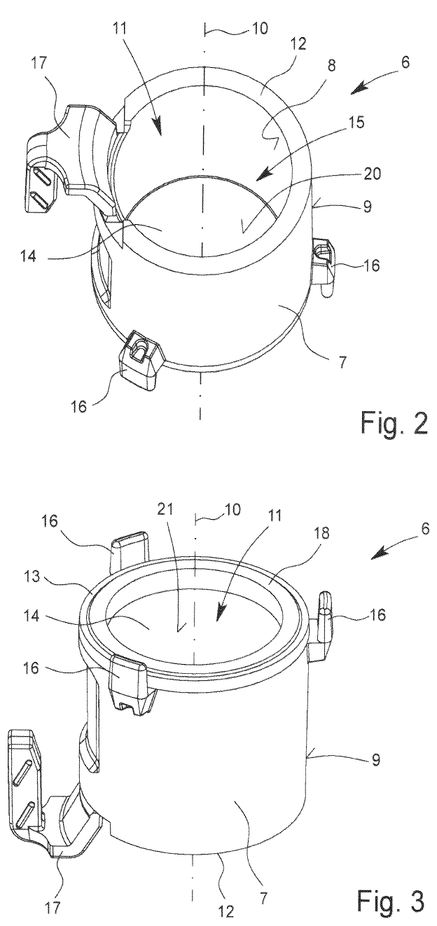

As shown, for example, in FIG. 2 and FIG. 3, the cell culture insert 6 according to the invention has a side wall 7 with an inner face 8 and an outer face 9, which extends essentially along an insert axis 10 and surrounds it completely, whereby the inner face 8 of the side wall 7 defines a cavity 11. In the preferred embodiment shown in the figures, the side wall 7 forms a hollow cylinder and surrounds the cavity 11 in a substantially hollow cylindrical manner. The side wall 7 extends along the insertion axis 10 from an upper opening at the front end, which is bounded by an opening rim 12, to a bottom opening at the front end, which is bounded by a bottom rim 13. In the area of the bottom opening, a bottom formed by a membrane 14 is arranged, which is connected to the side wall 7. The bottom in the form of a membrane 14 delimits on one side in the axial direction a cell culture space 15, which is delimited by the side wall 7 in the radial direction and in which cell growth takes place. Starting from the bottom in the form of a membrane 14, the cell culture space 15 extends in the axial direction up to the opening bounded by the opening rim 12.

In the present case, the term “axial” always refers to the insertion axis 10. The same applies to the terms “radial” and “circumferential direction”, which in the present case also always refer to the insertion axis 10.

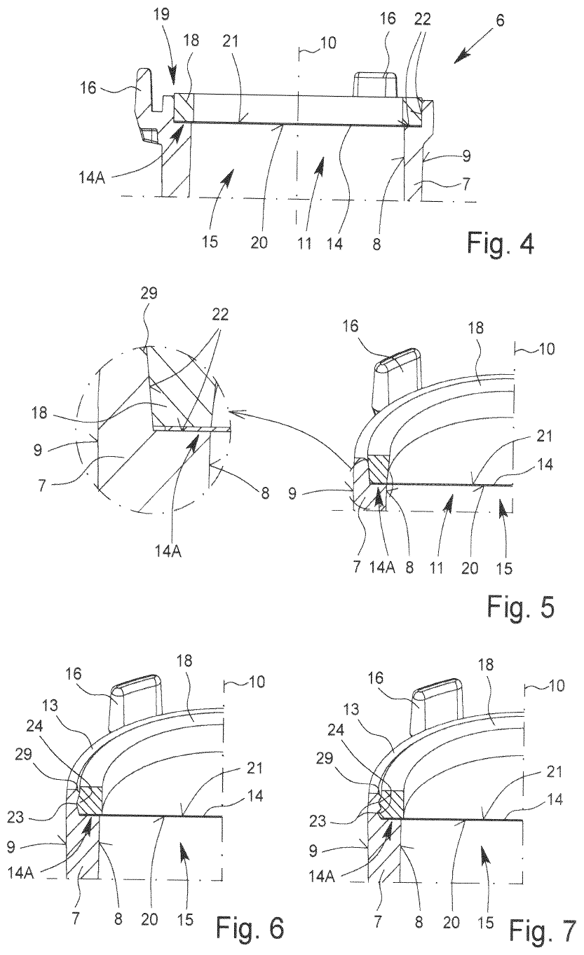

The bottom, which is designed as a membrane 14, can rest against the side wall 7 at the front and delimit it at the front. In the present case, the side wall 7 projects beyond the bottom in the form of a membrane 14 in the axial direction, as shown in FIG. 4 as an example.

On the outer face 9 of the side wall 7, a plurality of downwardly projecting feet 16, exactly three in the figures, are arranged (FIG. 2), with which the side wall 7 can be placed on a support with a small, uniform distance between the bottom, which is formed as a membrane 14, and the support so that it cannot tip over, as shown in FIG. 1 with reference to the wells 2 numbered B1 and B2. An outwardly projecting support arm 17 (FIG. 2) extends from the side wall 7 and can be placed on the well plate 1 in the rim area of a well 2, as also shown in FIG. 1 in relation to the wells 2 numbered A1 and A2.

It can be seen from FIG. 4 to FIG. 7 that the cell culture insert 6 according to the invention has a fastening element 18, wherein the membrane 14 is fastened to the inner face 8 of the side wall 7 by means of the fastening element 18 in a force-fit and/or form-fit manner. The membrane 14 is preferably arranged completely in the cavity 11.

In the present case, the term “friction-locked” means that movement between the two fastening partners, in the present case between the membrane 14 and the inner face 8 of the side wall 7, is prevented by static friction. Force-fit fastenings require a normal force acting on the connecting surfaces of the fastening partners. A mutual relative movement of the fastening partners is prevented as long as the counterforce caused by the static friction is not exceeded.

In the present case, it is preferably provided that the normal force acting between the membrane 14 and the side wall 7 is generated by a form-fit and/or force-fit connection between the fastening element 18 and the side wall 7. It is preferably provided that the fastening element 18 is in direct positive and/or non-positive engagement with the side wall 7, in particular with the inner face 8 of the side wall 7.

In the present case, the term “form-fit fastened” means the blocking or locking of a relative movement between the fastening partners, in the present case between the membrane 14 and the inner face 8 of the side wall 7, by means of design details. A positive fit can block relative movements in one or more directions or dimensions. A positive fit can be realized, for example, by interlocking the fastening partners or by interlocking one of the fastening partners with another component.

It is preferable for the fastening element 18 to engage directly with the side wall 7, in particular the inner face 8 of the side wall 7, in a form-fitting and/or force-fitting manner such that the membrane 18 is fastened in a form-fitting manner to the inner face 8 of the side wall 7. A form-fit fastening of the membrane 14 to the inner face 8 of the inner wall 7 is particularly suitable for dimensionally stable membranes 14. The form-fit fastening of the membrane 14 can also be advantageous if a particularly large radial expansion of the membrane 14 is to be made possible, as has already been described in detail above.

As shown in the figures, the membrane 14 is designed as a cylinder or cylindrical or disk-shaped. Other designs of the membrane 14 are also possible, for example, a square or rectangular base area transverse to the insertion axis 10, so that a shape of the membrane 14 adapted to the respective application can be used.

The membrane 14 is preferably formed in one piece. However, it is also possible for the membrane 14 to be in several parts or to consist of several individual components.

The membrane 14 can have a thickness of 5 μm to 500 μm, preferably of 10 μm to 200 μm, more preferably of at least substantially 100 μm.

In the present case, the term “thickness” refers to the extension of the membrane 14 along the insertion axis 10 in the attached state. Furthermore, the term “thickness” refers in particular to the dry membrane 14 before the membrane 14 comes into contact with nutrient fluid or the like.

Alternatively, the membrane 14 can also be manufactured as a fabric. The membrane 14 formed as a woven fabric can have a thickness of 10 μm to 1000 μm, preferably of 20 μm to 500 μm, further preferably of at least substantially 250 μm.

The membrane 14 preferably has an outer rim portion 14A. In particular, the rim portion 14A is circumferential in the circumferential direction and preferably completely surrounds the membrane 14 in the radial direction relative to the insertion axis 10.

In the fastened state, the rim portion 14A is preferably arranged between the inner face 8 of the side wall 7 and an outer face of the fastening element 18, as shown in FIG. 4 to FIG. 10.

The membrane 14 preferably has a top face 20 and a bottom face 21 which, in the fastened state, are arranged at least substantially transversely, in particular perpendicular to the insert axis 10. As FIGS. 4 to 10 further show, the membrane 14 rests with the top face 20 or the bottom face 21 of the membrane 14, in particular of the rim portion 14A, against the inner face 8 of the side wall 7 and with the other component of the top face 20 and bottom face 21 against the fastening element 18.

In the present case, the membrane 14 consists of at least one organic material, here and preferably of collagen.

Alternatively, the membrane 14 can be made of a material that cannot be bonded to the side wall 7. This can be the case, for example, with a material that is particularly temperature-sensitive and whose mechanical properties and permeability can be negatively impaired by correspondingly high temperatures. It also includes materials whose mechanical properties and permeability can be negatively affected by the use of suitable adhesives.

It is also possible for the membrane 14 to be made of a different material, in particular plastic. In this context, reference may be made to the introductory description.

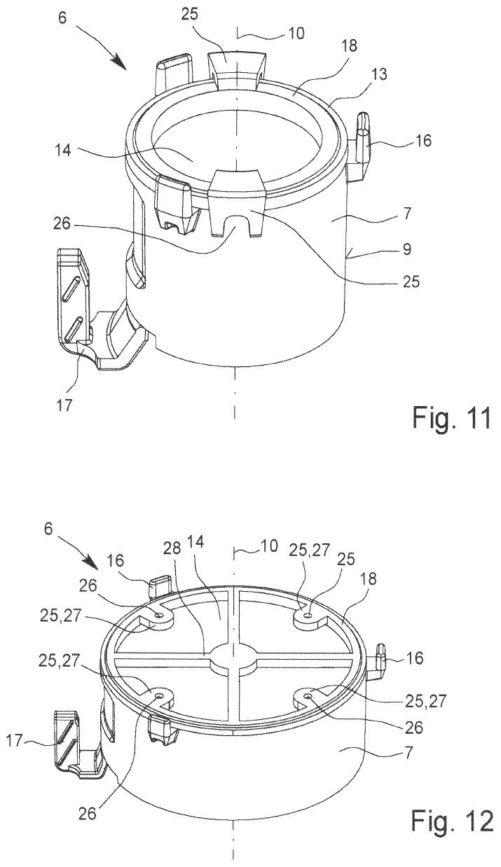

In FIG. 4 to FIG. 8, it can be seen that the fastening element 18 is arranged at least partially in the cavity 11. Alternatively or additionally, it is possible for the fastening element 18 to protrude outwards beyond the side wall 7 at least in sections (FIG. 11).

In the present case, the fastening element 18 is pressed against the inner face 8 of the side wall 7. The fastening element 18 has a pressing force acting radially outwards on the inner face 8 of the side wall 7. The fastening element 18 preferably forms an interference fit with the side wall 7. The fastening element 18 is thus pressed into the opening of the side wall 7, which is delimited by the bottom rim 13, in a particularly simple manner.

The fastening element 18 is designed as a hollow cylinder, although other constructive designs, such as a cylindrical or disk-shaped design, are also possible. In the embodiment shown in FIG. 4, the fastening element 18 has a rectangular longitudinal section. Other shaped longitudinal sections are also possible. For example, in the embodiment shown in FIG. 5, the fastening element 18 has a conical longitudinal section.

In the embodiments shown in the figures and preferred in this respect, the fastening element 18 is formed in one piece. The fastening element 18 has an outer diameter and an inner diameter, the term “outer diameter” in the present case being understood to mean the minimum outer diameter of the fastening element 18 and the term “inner diameter” being understood to mean the maximum inner diameter of the fastening element 18. As shown in FIG. 4, the outer diameter of the fastening element 18 is larger than the diameter of the membrane 14. The inner diameter of the fastening element 18 is additionally or alternatively smaller than the diameter of the membrane 14. The term “diameter” is clearly defined for disc-shaped or cylindrical membranes 14 and hollow cylindrical fastening elements 18. If the membrane 14 does not have a circular base and/or the fastening element 18 does not have a circular or ring-shaped base, the term “diameter” in the present case means the maximum extension in the radial direction. The membrane 14 can rest against the fastening element 18 in an axial direction and in this way be fixed in an axial direction by the fastening element 18, as shown in FIG. 4.

The fastening element 18 has polypropylene, polyethylene, polystyrene and/or polylactide acid or consists of polypropylene, polyethylene, polystyrene and/or polylactide acid, whereby a design made of other materials, preferably one or more plastics, is also possible. A low modulus of elasticity can ensure an elastic material behavior of the fastening element 18 and an associated seal between the fastening element 18 and the side wall 7.

In order to increase the static friction and/or the seal between the side wall 7 and the fastening element 18, the fastening element 18 can have a roughened surface on an outer face.

As shown in particular in FIG. 4, a preferably circumferential stop 19 for fastening the membrane 14 is formed on the inner face 8 of the side wall 7, the fastening element 18 is pressed into the stop 19 and the fastening element 18 holds or presses the membrane 14, in particular via the rim portion 14A, against the stop 19. In the embodiment shown in the figures, it is provided that the fastening element 18 presses the membrane 14 against the side wall 7 in the axial direction. The membrane 14, in particular the rim portion 14A, is then arranged between the stop 19 and the fastening element 18 and preferably pressed in between these.

The membrane 14 has an top face 20 and a bottom face 21, whereby the membrane 14 preferably rests against the stop 19 exclusively with a section of its top face 20. Particularly uniform pressing of the membrane 14 against the stop 19 can be achieved if the membrane 14 preferably rests against the fastening element 18 exclusively with a section of its bottom face 21, as shown in FIG. 4. The stop 19 then fixes the membrane 14 in a first axial direction, while at the same time the fastening element 18 fixes the membrane 14 in the opposite, second axial direction.

FIGS. 4 to 8 show, as a further special feature of the cell culture insert 6 according to the invention, that the stop 19 is formed by a recess on the inner face 8 of the side wall 7 and has two stop surfaces 22 arranged at an angle, in particular at right angles, to one another, preferably circumferentially, preferably with one stop surface 22 extending at least substantially in the radial direction and in the circumferential direction and the other stop surface 22 extending at least substantially in the axial direction and in the circumferential direction. If the two stop surfaces 22 are fully formed, it is possible for the membrane 14 to rest with an rim portion fully against one stop surface 22, whereby uniform pressing-in of the membrane 14 can be achieved.

As shown in particular in the detailed view of FIG. 5, the membrane 14, in particular the rim portion 14A of the membrane 14, is in contact with one stop surface 22, preferably the stop surface 22 extending substantially in the radial direction and in the circumferential direction, and is spaced from the other stop surface 22, preferably the stop surface 22 extending substantially in the axial direction and in the circumferential direction. The membrane 14 is then fixed in the axial direction between the fastening element 18 and the stop 19. As can also be seen in the detailed view of FIG. 5, the membrane 14 has a distance from the inner face 8 of the side wall 7 in the radial direction. If the membrane 14 expands, for example due to contact with a liquid, the membrane 14 can expand in the radial direction without bulging in the axial direction. In this way, a uniform, even clamping of the membrane 14 is made possible, whereby a curvature of the membrane 14 in the axial direction is effectively prevented.

In order to produce a positive connection between the fastening element 18 and the side wall 7, the embodiment shown in FIG. 6 and FIG. 7 provides that the stop 19 has at least one recess 23 on the stop surface 22 extending essentially in the axial direction and in the circumferential direction and the fastening element 18 has at least one complementary protrusion 24 on an outer face and that the recess 23 and the protrusion 24 are in engagement or form a positive fit in the fastened state. In FIG. 6, the stop 19 has precisely one recess 23 on the stop surface 22, which extends essentially in the axial direction and in the circumferential direction, and the fastening element 18 has a complementary protrusion 24.

FIG. 7 shows a further embodiment in which the stop 19 has two recesses 23 and the fastening element 18 has two complementary protrusions 24 on the stop surface 22, which runs essentially in the axial direction and in the circumferential direction. A reverse arrangement is also conceivable, in which the fastening element has a recess 23 and the stop 19 has a complementary protrusion 24.

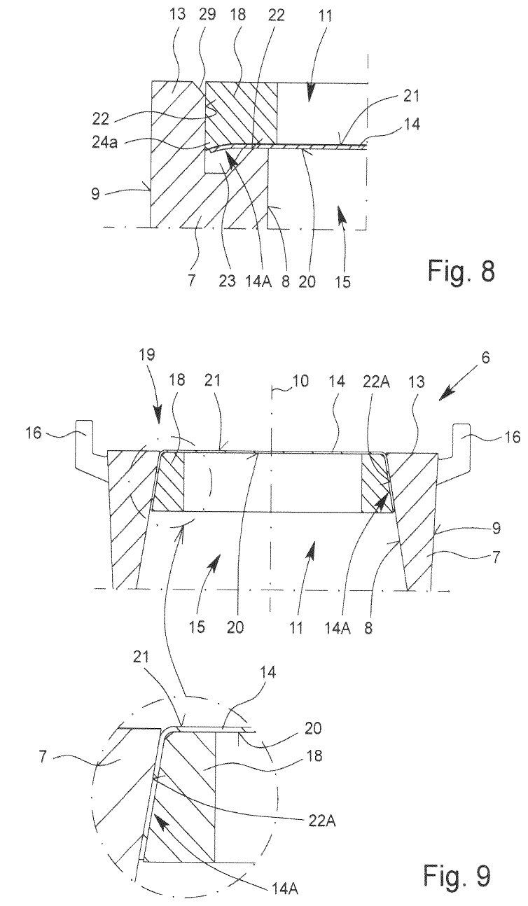

In the preferred embodiment shown in FIG. 8, the stop 19 has at least one recess 23 on the stop surface 22, which extends essentially in the radial direction and in the circumferential direction, and the fastening element 18 has at least one protrusion 24a on one end face, the protrusion 24a engaging at least in sections in the recess 23 and bracing the membrane 14 in the axial direction. The recess 23 is preferably provided between the stop surface 22, which extends substantially in the radial direction and in the circumferential direction, and the stop surface 22 recess 23, which extends substantially in the axial direction and in the circumferential direction. In the embodiment shown in FIG. 8, the fastening element 18 has the protrusion 24a on one end face, which extends essentially in the axial direction. In the assembled state of the cell culture insert 6, the protrusion 24a of the fastening element 18 causes additional tensioning of the membrane 14 in the region of its outer rim in the axial direction, as a result of which expansion of the membrane 14 on contact with the nutrient fluid can be further reduced in an advantageous manner. In the preferred embodiment shown in FIG. 8, the protrusion 24a is not complementary to the recess 23. However, it is also possible to form the protrusion 24a complementary to the recess 23.

In order to reliably seal the connection between the fastening element 18 and the stop 19 in particular, the fastening element 18 and/or the stop 19 of the side wall 7 can have grooves or a sealing lip. Corresponding grooves or the sealing lip are preferably made of a plastic, in particular an elastomer. The grooves or the sealing lip can also be formed from several plastics, in particular copolymers.

A further special feature of the cell culture insert 6 according to the invention is that the fastening element 18 and the membrane 14 can be detached from the side wall 7, which makes it easy to replace the membrane 14 by means of a modular structure of the cell culture insert 6. The membrane 14 can then be replaced with another membrane 14 adapted to the application in a particularly simple manner.

In the embodiments of the cell culture insert 6 shown in FIG. 3 to FIG. 8, the fastening element 18 can be pushed or pressed into the bottom opening of the side wall 7, which is delimited by the bottom rim 13, for assembly purposes.

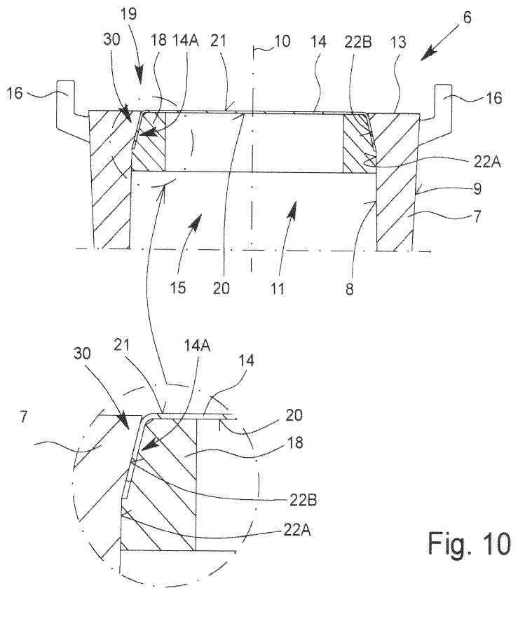

FIG. 9 and FIG. 10 each show a longitudinal section of a cell culture insert 6 according to a respective further embodiment. The further embodiments differ from the embodiments shown in FIG. 3 to FIG. 8 in particular in that the fastening element 18 and/or the membrane 14 can be pushed or pressed into the opening of the side wall 7 delimited by the opening rim 12 for assembly purposes. In particular, the fastening element 18 together with the membrane 14 can be pushed or pressed into the opening defined by the opening rim 12 for assembly purposes.

Preferably, the side wall 7 in the embodiments shown in FIGS. 9 and 10 is formed at least in sections as a hollow truncated cone. In particular, the side wall 7 or a section of the side wall 7 surrounds the cavity 11 essentially in the shape of a hollow truncated cone.

Preferably, the side wall 7 is formed as a truncated hollow cone over its entire axial extent. The cavity 11 is then shaped in particular as a truncated cone or truncated cone.

The side wall 7, in particular the inner face 8 of the side wall 7, preferably forms an angle to the insert axis 10, whereby the angle is in particular at least 0.5°, preferably at least 3°, and/or less than 10°, preferably less than 8°.

The cavity 11 then narrows preferably starting from the opening bounded by the opening rim 12 towards the bottom opening bounded by the bottom rim 13, in particular conically at an angle of at least 0.5° and/or less than 10° relative to the insertion axis 10. However, it is also possible that the cavity 11 tapers conically only in sections along the insertion axis 10 in the direction of the opening bounded by the bottom rim 13.

In both of the aforementioned cases, the cavity 11 has a uniform or constant taper along the insert axis 10.

The unmounted or unattached membrane 14 can be disk-shaped or cylindrical. In particular, the membrane 14 can have a larger diameter than the fastening element 18. It is alternatively or additionally possible for the membrane 14 to be preformed, as FIG. 9 and FIG. 10 show. In particular, the membrane 14 can be shaped like a cup.

As FIG. 9 and FIG. 10 further show, the fastening element 18 preferably has an outer face. In particular, the outer face has an outer radial contour that is complementary to at least one section of the side wall 7. In particular, the radial contour of the fastening element 18 is complementary to at least one section of the inner face 8 of the side wall 7.

The membrane 14 is preferably dimensioned such that in the assembled state of the cell culture insert 6 shown in FIG. 9, the outer face, in particular the radial contour, of the fastening element 18 is covered at least in sections by the membrane 14.

The side wall 7 preferably forms a stop surface 22A, against which the membrane 14, in particular the rim portion 14A, rests in the fastened state. As FIG. 10 shows, the membrane 14 or the rim portion 14A rests against the stop surface 22A, in particular with the bottom face 21. The fastening element 18 preferably lies with an outer face, in particular its radial contour, against the top face 20 of the membrane 14 and clamps the membrane 14 against the stop surface 22A. In this way, the membrane 14 and the fastening element 18 are fixed in the cell culture insert 6.

In the fixed state, the membrane 14 shown in FIGS. 9 and 10 is preferably arranged at least substantially at the height of the bottom rim 13 delimiting the end face of the cavity 11. In the present case, the term “height” is preferably to be understood as a plane that extends at least substantially perpendicular to the insertion axis 10. In the present case, the term “essentially” in relation to the height preferably means that there may be a dimensional deviation in the axial direction of up to 1.5 mm, preferably up to 1.2 mm, more preferably up to 1 mm, between the bottom rim 13 and the membrane 14 forming the bottom of the cell culture insert 6.

In this way, the membrane 14 can form a plane with the bottom rim 13, which delimits the side wall 7 in the axial direction, taking into account the above-mentioned deviation in the axial direction and delimit the cell culture insert 6 on the end face.

The radial contour of the fastening element 18 can have grooves in order to improve the force-fit and/or form-fit fastening of the fastening element 18 and/or membrane 14 to the inner face 8 of the side wall 7.

Alternatively or additionally, it is possible for the inner face 8 to have at least one recess 23 on the stop surface 22A and for the fastening element 18 to have at least one complementary protrusion 24 on an outer face, as has already been explained with reference to FIG. 6 and FIG. 7. In the fastened state, the protrusion 24 can engage in the recess in order to create a positive engagement between the fastening element 18 and the side wall 7.

For assembly purposes, the fastening element 18 can be pushed or inserted into the opening of the side wall 7 defined by the opening rim 12, as already explained above. The fastening element 18 can be pushed in or inserted together with the membrane 14.

The fastening element 18 can be pushed or inserted along the insertion axis 10 into the cavity 11 formed by the side wall 7 until the membrane 14 is pressed in between the radial contour of the fastening element 18 and the inner face 8 of the side wall 7, in particular the stop surface 22A. In the fastened state, the fastening element 18 can also bear against a stop surface 22A of the side wall 7, in particular with its radial contour.

For disassembly, the fastening element 18 can be pressed or pushed along the insertion axis 10 out of the cavity 11 through the opening of the side wall 7 bounding the opening rim 12.

It has been shown that the membrane 14 permanently wraps around the fastening element 18 when moistened with nutrient fluid, so that a unit consisting of membrane 14 and fastening element 18 is formed. The membrane 14 can thus be removed from the cell culture insert 6 together with the fastening element 18 in a particularly simple manner.

The unit comprising membrane 14 and fastening element 18 can be easily handled manually and/or with the aid of a tool, for example tweezers or the like. The top surface 20 of the membrane 14, which is colonized with cells, can then be examined optically using a microscope, in particular an incident light microscope. It is then not necessary to cut the membrane 14 or parts of the membrane 14 out of the cell culture insert 6, whereby cells can be destroyed.

According to the prior art, it is possible to determine whether the membrane 14 is sufficiently colonized with cells by means of an electrical resistance measurement. Due to the simple and non-destructive removal of the membrane 14 from the cell culture insert 6, it is possible to determine optically, for example using a microscope, whether the membrane 14 is sufficiently colonized with cells or with a sufficient number of cells.

FIG. 10 shows a longitudinal section of a cell culture insert 6 according to a further embodiment. The side wall 7 of the cell culture insert 6 shown in FIG. 10 preferably has a stop 19 for clamping the membrane 14. The stop 19 is preferably formed as a clamping protrusion 30 on the inner face 8 of the side wall 7. In particular, the clamping protrusion 30 extends from the side wall 7 inwards in a radial direction. In particular, the clamping protrusion 30 is arranged in the area of the bottom rim 13 of the side wall 7 and/or merges into the bottom rim 13.

In particular, the clamping protrusion 30 has a stop surface 22B against which the membrane 14 rests in the fastened state. The stop surface 22B preferably extends transversely to the radial direction and/or to the axial direction. The stop surface 22B on the clamping protrusion 30 is advantageously shaped circumferentially in such a way that the clamping protrusion 30 is conical in longitudinal section, as FIG. 10 shows.

In particular, the stop surface 22B forms an angle to the insert axis 10, wherein the angle is at least 10°, preferably at least 15°, and/or less than 45°, preferably less than 35°.

As FIG. 9 shows, in the fastened state the membrane 14 preferably rests with its bottom face 21 against the stop surface 22B, which is arranged transversely to the radial and/or axial direction, on the clamping protrusion 30. The fastening element 18 rests with an outer face against the membrane 14, in particular the top face 20, and clamps the membrane 14 against the stop surface 22B.

As FIG. 9 further shows, the fastening element 18 preferably has an outer face that has an outer radial contour and is shaped to complement the clamping protrusion 30. In the fastened state, the fastening element 18 can also bear with its outer face against the inner face 8 of the side wall 7, in particular against the stop surface 22A formed by the side wall 7. The stop surface 22A formed by the side wall 7 and the stop surface 22B formed by the clamping protrusion 30 are preferably arranged at an angle to each other. The two stop surfaces 22A, 22B preferably form an angle of at least 110° and/or less than 178°.

The radial contour can have grooves in order to improve the force-fit and/or form-fit fastening of the fastening element 18 and/or membrane 14 to the inner face 8 of the side wall 7 and/or to the clamping protrusion 30.

Alternatively or additionally, it is possible for the stop 19 to have at least one recess 23 on the stop surface 22B and for the fastening element 18 to have at least one complementary protrusion 24 on an outer face, in particular its radial contour, as has been explained with reference to FIG. 6 and FIG. 7. In the fastened state, the protrusion 24 can engage in the recess in order to produce a positive engagement.

As already mentioned, the fastening element 18 can be pushed or inserted into the opening of the side wall 7 delimited by the opening rim 12 for assembly purposes. In order to facilitate easy assembly, the cavity 11 can narrow from the opening defined by the opening rim 12 towards the bottom opening defined by the bottom rim 13, in particular conically at an angle of 0.5° to 10°. In this way, a reliable, at least force-fit fastening between the side wall 7 and the fastening element 18 can be ensured.

In the preferred embodiment shown in FIGS. 11 and 12, it is provided that the fastening element 18 has at least one removal element 25 for manual removal and/or removal by means of a tool. The removal element 25 can be used to simplify the removal of the fastening element 18 by improving the application of force or by forming a lever arm for the user. The removal element 25 can extend in a radial and/or axial direction.

Furthermore, it is particularly advantageous if the fastening element 18 has at least one externally accessible indentation 26 for removal by means of a tool. The tool can engage at least partially in the indentation 26 and simplify the removal of the fastening element 18 by improving the application of force or by forming a lever arm for the user. In the preferred embodiment shown in FIG. 11 and FIG. 12, the removal element has the indentation 26.

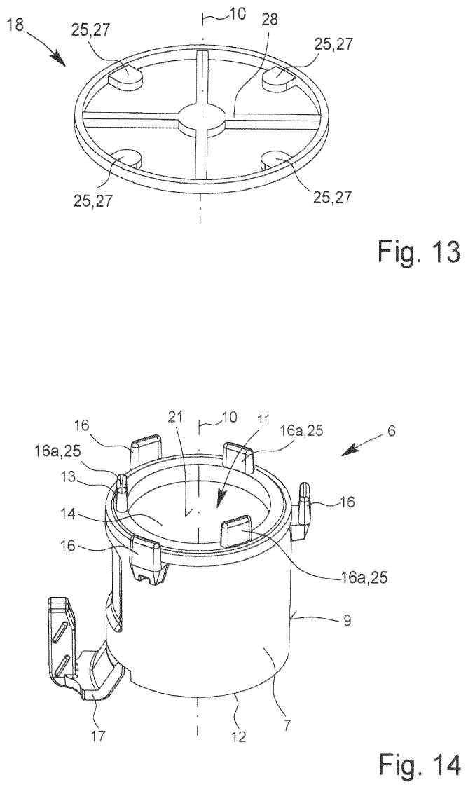

In FIG. 12, it is shown by way of example that the fastening element 18 has at least one protrusion 27, four in FIG. 12, projecting radially inwards from an inner face of the fastening element 18 with a indentation 26, accessible from the outside and substantially axially aligned, for removal by means of a tool. It is alternatively or additionally possible for the protrusion 27 to be spaced from a rim of the inner face of the fastening element 18, as shown in detail in FIG. 13.

Alternatively, it is also possible to remove the fastening element 18 using a so-called lock-and-key principle, in which a tool, for example in the form of a key such as a Torx or wrench or a screwdriver, can be used to release a locking mechanism, in particular a positive locking mechanism, between the fastening element 18 and the side wall 7.

he fastening element 18 can be designed to be ferromagnetic for removal by means of a magnetic device. With the aid of a permanent magnet or an electromagnet, it is then possible to exert a force on the fastening element 18 to remove it.

In the embodiment shown in the figures, the fastening element 18 is formed in one piece. However, it is also possible for the fastening element 18 to be designed in several parts, preferably in two parts. It is then possible for the membrane 14 to be clamped between the parts of the fastening element 18.

It is particularly advantageous if the fastening element 18 is made in several parts, preferably in two parts, and the parts of the fastening element 18 can be fixed together. In this way, a pre-assembled functional unit consisting of the membrane 14 and the fastening element 18 can be obtained. “Pre-assembled” in the present case means that the individual components are already fixed together as a unit. The term “functional unit” means that the pre-assembled unit has all the components required for the respective functionality. In the present case, the term “functional unit” therefore means that it has all the components required for non-positive and/or positive attachment of the membrane 14 to the side wall 7. The fastening element 18 can then be inserted into the bottom opening of the side wall 7 together with the membrane 14 as a pre-assembled functional unit in a manner to be explained below and pressed in against the stop 19.

It is also possible to design the fastening element 18 in several parts in the circumferential direction. The parts of the fastening element 18 can then be inserted simultaneously or successively into the bottom opening of the side wall 7 and pressed in against the stop 19 in order to fasten the membrane 14 to the side wall 7 in a force-fit or form-fit manner.

In particular with large diameters of the cell culture insert 6 and/or with particularly sensitive membranes 14, it is imperative for cell cultivation to prevent curvature and/or damage to the membrane 14. In the embodiment shown in FIG. 12, provision is made for the fastening element 18 to have a support device 28, which is arranged below the membrane 14. The support device 28 also provides for a stiffening of the fastening element 18, whereby the membrane 14 can be clamped particularly evenly and the probability of a curvature of the membrane 14 can be reduced. A curvature or sagging of the membrane 14 can be prevented particularly effectively if the membrane 14 rests with its bottom face 21 against the support device 28.

It is possible that at least three support elements 16a are attached to a bottom face of the fastening element 18 for setting up the cell culture insert 6 so that it cannot tip over and for removing the fastening element 18, as FIG. 14 shows. The support elements 16a are thus designed as a multifunctional part which, on the one hand, enables the cell culture insert 6 to be set up so that it cannot tip over and, on the other hand, serves to remove the fastening element 18. The support elements 16a are thus designed as removal elements 25. As can be seen from FIG. 11, it is possible for the cell culture insert 6 to be placed together on the support elements 16a and the feet 16 on a base. In this way, the cell culture insert 6 can be effectively prevented from tipping over. When using a fastening element 18 with support elements 16a, it is also possible to use a cell culture insert 6 without feet 16. It is then still possible to use the support elements 16a to place the cell culture insert 6 on a support with a small, uniform distance between the membrane 14 and the support so that it cannot tip over.

Reference is made to FIG. 4 for a description of how the connection between the side wall 7 and the membrane 14 is made. The membrane 14 is first placed on the essentially radially extending and circumferentially circumferential stop surface 22. The fastening element 18 is then inserted into the bottom opening of the side wall 7 in an assembly movement, particularly in the axial direction, and pressed against the stop 19, particularly in the axial direction. A pressure punch of a tool not shown, in particular a tool of a machine tool, can be used for this purpose. In addition to being easy to perform, mechanical pressing has the advantage that force values can be specified in a particularly simple manner in order to achieve optimum pressing between the side wall 7 and the fastening element 18.

The fastening element 18 is pressed against the stop 19 with a contact pressure of 30 N to 1000 N. For example, with an oversize of the outer face of the fastening element 18 of 0.05 mm compared to the stop surface 22, which essentially runs in the radial direction and in the circumferential direction, the fastening element 18 can be pressed against the stop 19 with a contact pressure of 30 N to 300 N, preferably from 40 N to 250 N, preferably from 50 N to 200 N. If the outer face of the fastening element 18 is 0.1 mm larger than the stop surface 22, which essentially runs in a radial and circumferential direction, the fastening element 18 can be pressed against the stop 19 with a contact pressure of 300 N to 1000 N, preferably 400 N to 850 N, preferably 500 N to 700 N.

If the fastening element 18 has support elements 16a, the plunger can have corresponding recesses to accommodate the support elements 16a. It is then possible that the force generated by the plunger on the fastening element 18 does not act on the support elements 16a.

It is also possible that the force generated by the pressure stamp also or exclusively acts on the support elements 16a.

In a further and in this respect preferred embodiment, it is also possible to insert the membrane 14 together with the fastening element 18 into the bottom opening of the side wall 7 until the membrane 14 abuts against the stop surface 22 extending substantially in the radial direction and running around in the circumferential direction and/or the fastening element 18 is pressed in against the stop 19.

Insertion of the fastening element 18 into the opening of the side wall 7 bounded by the bottom rim 13 can be facilitated if the transition from the inner face 8 of the side wall 7 to the bottom rim 13 of the side wall 7 is formed by a chamfer 29. In this way, the mechanical stress between the bottom rim 13 of the side wall 7 and the end face of the fastening element 18 facing the cell culture space 15 in the assembled state can be reduced. Alternatively or additionally, the transition from the end face of the fastening element 18 facing the cell culture space 15 and the outer face of the fastening element 18 can be formed by a chamfer 29. The chamfer angle is between 35° and 55°, preferably substantially 45°, as shown in FIG. 4.

The cell culture insert 6 can be delivered fully assembled. Due to the modular structure, it is also possible to deliver individual components of the cell culture insert 6, such as the fastening element 18, the membrane 14 and/or the side wall 7, individually or in any number and/or combination with other components of the cell culture insert 6. In the present case, the membrane 14 is preferably designed as a stamped part, which enables particularly simple, in particular mechanical, manufacture of the membrane 14 by means of a stamping process.

In an alternative and thus preferred embodiment, the fastening element 18 is intended to be connected to the side wall 7 in a form-fitting and/or material-fitting manner. The fastening element 18 can be inserted into the bottom opening of the side wall 7 and fixed relative to the side wall 7 and/or to the side wall 7, in particular in an axial and/or radial direction, in a form-fitting manner. For example, the fastening element 18 can have at least one, in particular protruding, positive-locking element and the side wall 7 can have a positive-locking counter element that can be brought into engagement with the positive-locking element, in particular protruding.

It is then additionally or alternatively possible to create a material connection, in particular an essentially punctual connection, between the form-fit element and the form-fit mating element in order to securely fix the fastening element 18 to the side wall 7. It is particularly advantageous if the membrane 14 does not come into contact with any adhesive that may be used. Due to the essentially punctual form-fit connection, only a small amount of adhesive is required. It is then also possible to use adhesives that are only toxic to the cell culture and/or the culture medium in larger quantities. Preferably, the adhesive used is not soluble in the culture medium in order to prevent any negative impact on the culture medium, the cell cultures and/or the membrane. The punctual connection can also be created by thermally bonding the form-fit element to the form-fit counter element.

To detach the fastening element 18 from the side wall 7, it is necessary to break the form-fit connection. Due to the essentially punctual material connection and the associated small connection surface, this is possible in a particularly simple manner with little effort. The detachment can also be carried out by cutting off or cutting off the form-fit elements and form-fit mating elements that are connected to each other with a positive fit. The fastening element 18 can then be removed, in particular together with the membrane 14.

All the aspects of the invention described above can be used both individually and in combination with one another.

### REFERENCE NUMBER LIST

1 Well plate
2 Well of 1
3 Face of 1
4 Wall of 2
5 Rim of 2
6 Cell culture insert
7 Side wall of 6
8 Inner face of 7
9 Outer face of 7
10 Insert axis
11 Cavity of 6
12 Opening rim of 7
13 Bottom rim of 7
14 Membrane of 6
14A Rim portion of 14
15 Cell culture space of 6
16 Feet of 6
16a Support element of 18
17 Support arm of 6
18 Fastening element of 6
19 Stop of 7
20 Top face of 14
21 Bottom face of 14
22 Stop surface of 19
22A Stop surface of 7
22B Stop surface of 30
23 Recess of 22
24 Protrusion of 18
24a Protrusion of 18
25 Removal element of 18
26 Indentation of 27
27 Protrusion of 18
28 Supporting device of 18
29 Chamfer of 7 or 18
30 Clamping protrusion of 7

1-16. (canceled)

17. A cell culture insert for cultivating cells, comprising
a side wall which encloses a cavity and comprises an inner face, which delimits the cavity, and an outer face,
a bottom which is a membrane, and
a fastening element,
wherein the membrane is fastened to the inner face of the side wall by the fastening element in at least one of a force-fitting or form-fitting manner.

18. The cell culture insert according to claim 17, wherein the side wall at least one of forms a hollow cylinder or surrounds the cavity in a substantially hollow-cylindrical manner.

19. The cell culture insert according to claim 17, wherein the membrane at least one of:
is arranged in the cavity,
is shaped as a cylinder or disk,
comprises a radially outer rim portion, which is arranged between the inner face of the side wall and an outer face of the fastening element,
is arranged at least substantially at the level of a bottom rim of the side wall, the bottom rim delimiting the cavity on an end face, or
is formed from at least one organic material or from at least one material that cannot be bonded to the side wall.

20. The cell culture insert according to claim 17, wherein the fastening element at least one of:
is arranged at least partially in the cavity,
protrudes outwards beyond the side wall,
is pressed against the inner face of the side wall,
has a pressing force acting radially outwards on the side wall,
forms a press fit with the side wall,
is formed in one piece,
is shaped as a hollow cylinder,
has an outer diameter that is larger than the diameter of the membrane,
has an inner diameter that is smaller than the diameter of the membrane,
comprises polypropylene, polyethylene, polystyrene and/or polylactide acid or consists of polypropylene, polyethylene, polystyrene and/or polylactide acid, or
has a roughened surface on an outer face of the fastening element.

21. The cell culture insert according to claim 17, wherein the side wall at least one of forms a hollow truncated cone or surrounds the cavity substantially in the shape of a hollow truncated cone,
wherein an outer face of the fastening element is formed complementary to the side wall delimiting the cavity or the inner face of the side wall, and
wherein the fastening element holds the membrane or is pressed against the inner face.

22. The cell culture insert according to claim 21, wherein the inner face of the side wall comprises at least one stop surface and wherein the membrane lies against the stop surface.

23. The cell culture insert according to claim 17, wherein
a stop for fastening the membrane is formed on the inner face of the side wall,
the fastening element is press-fit at the stop, and
the fastening element holds the membrane or presses it against the stop.

24. The cell culture insert according to claim 23, wherein the membrane comprises a top face and a bottom face and wherein the membrane lies against the stop exclusively with a portion of its top face or exclusively with a portion of its bottom face.

25. The cell culture insert according to claim 23, wherein the stop is formed by a recess on the inner face of the side wall and comprises two stop surfaces arranged at an angle to one another.

26. The cell culture insert according to claim 25, wherein the membrane lies against one stop surface of the two stop surfaces and is spaced apart from another stop surface of the two stop surfaces.

27. The cell culture insert according to claim 25, wherein a first stop surface of the two stop surfaces extends at least substantially in radial direction and in circumferential direction and a second stop surface of the two stop surfaces extends at least substantially in axial direction and in circumferential direction.

28. The cell culture insert according to claim 27, wherein at least one of:
the stop comprises at least one recess on the second stop surface, and the fastening element comprises at least one complementary protrusion on an outer face of the fastening element, and wherein the recess and the protrusion are in engagement and/or form a positive fit in the fastened state, or
the stop comprises at least one recess on the first stop surface and the fastening element comprises at least one protrusion on an end face and wherein the protrusion engages at least in sections in the recess and braces the membrane in the axial direction.

29. The cell culture insert according to claim 24, wherein the stop is formed by a clamping protrusion on the inner face of the side wall and comprises a circumferential stop surface, wherein the membrane lies against the stop surface.

30. The cell culture insert according to claim 29, wherein at least one of:
the stop comprises at least one recess on the stop surface and the fastening element comprises at least one complementary protrusion on an outer face of the fastening element, the recess and the protrusion being in engagement or forming a positive fit in the fastened state, or
at least one portion of the fastening element lies against the inner face of the side wall.

31. The cell culture insert according to claim 17, wherein at least one of the fastening element or the side wall has grooves or a sealing lip.

32. The cell culture insert according to claim 17, wherein the fastening element and the membrane are detachable from the side wall.

33. The cell culture insert according to claim 32, wherein at least one of:
the fastening element comprises at least one removal element for at least one of manual removal or removal by means of a tool,
the fastening element comprises at least one externally accessible indentation for removal by means of a tool,
the fastening element comprises at least one protrusion projecting radially inwards from an inner face of the fastening element with an externally accessible indentation for removal by means of a tool, or
the fastening element is ferromagnetic for removal by means of a magnetic device.

34. The cell culture insert according to claim 17, wherein the fastening element is designed in multiple parts and the membrane is clamped between parts of the fastening element.

35. The cell culture insert according to claim 17, wherein the fastening element comprises a supporting device which is arranged below the membrane.

36. The cell culture insert according to claim 17, wherein at least three support elements are attached to a bottom face of the fastening element for tilt-proof positioning of the cell culture insert and for removing the fastening element.
